(19) United States
(12) Patent Application Publication
(10) Pub. No.: US 2025/0099518 A1
(43) Pub. Date: Mar. 27, 2025

(54) PROBIOTIC SINUS IRRIGATION COMPOSITION AND METHODS

(71) Applicant: THE BOARD OF TRUSTEES OF THE UNIVERSITY OF ILLINOIS, URBANA, IL (US)

(72) Inventors: VICTORIA S. LEE, CHICAGO, IL (US); JULIANA THEORELL, CHICAGO, IL (US)

(73) Assignee: THE BOARD OF TRUSTEES OF THE UNIVERSITY OF ILLINOIS, URBANA, IL (US)

(21) Appl. No.: 18/894,095

(22) Filed: Sep. 24, 2024

Related U.S. Application Data
(60) Provisional application No. 63/540,188, filed on Sep. 25, 2023.

(51) Int. Cl. A61K 35/747; A61K 9/00; A61K 35/00; A61K 35/744; A61P 11/02

(52) U.S. Cl. CPC A61K 35/747; A61K 9/0043; A61K 35/744; A61P 11/02; A61K 2035/115

(57) ABSTRACT

Disclosed are sinus irrigation compositions and pre-mix compositions comprising a Lactobacillales bacterium such as Lactobacillus sakei and kits containing the same. Also provided are methods of treating sinusitis in a subject by administering to the subject an effective amount of a composition comprising a Lactobacillales bacterium such as Lactobacillus sakei.

### INTRODUCTION

This application claims the benefit of priority from U.S. Provisional Application Ser. No. 63/540,188, filed Sep. 25, 2023, the contents of which are incorporated herein by reference in their entireties.

### BACKGROUND

Chronic rhinosinusitis (CRS) is a common chronic disease, affecting 13.8% of the United States population and accounting for over $20 billion in direct/indirect costs annually. It is an inflammatory disease of the sinonasal cavity. Patients suffer from some combination of cardinal sinonasal symptoms, specifically nasal obstruction, congestion, and/or blockage; anterior or posterior (mucopurulent) nasal drainage; loss or decreased sense of smell; and facial pressure, pain, and/or fullness. Also commonly reported include symptoms of severe fatigue and sleep problems including poor sleep quality, decreased cognitive function, and depression-type symptoms. Collectively, CRS has profound effects on functional well-being and general health-related quality of life. Patients tend to require long-term management and can experience exacerbations throughout their lives.

Standard treatment includes medical therapy and endoscopic sinus surgery (ESS). The most effective current medical treatment is oral corticosteroids, but their use is limited by significant side effects associated with chronic/recurrent courses, including bone loss/osteoporosis, avascular joint necrosis, cataracts, glaucoma, high blood pressure, high blood sugar, mood changes, stomach ulcers, electrolyte abnormalities, and menstrual irregularities. Additional medical treatments include oral antibiotics, leukotriene modifiers, and immunotherapy, systemic therapies that can be expensive with the potential for significant side effects, as well as corticosteroids in topical form.

Approximately 30% of patients, however, have recalcitrant symptoms/disease. There is an urgent need for novel treatments for these patients.

Dysregulation of the commensal microbiome is also believed to be at play in the sinonasal cavity (Ramakrishnan et al. (2013) Int. Forum Allergy Rhinol. 3(4):267-271). An in vitro study has found that Lactobacilli were depleted in CRS versus healthy subjects (Abreu et al. (2012) Sci. Transl. Med. 4 (151):151ra124). Nasal sprays, buccal tablets, buccal sprays, aerosols, throat lozenges and ear drops including probiotic Lactobacillus sp. have been described for application to external nares of the nostrils in the prevention and/or treatment of infections of the oronasopharyngeal cavity (WO 2015/140299 A1). In a murine model of sinusitis, Lactobacillus sakei defended against infection of pathogenic bacteria identified in the initial analyses, supporting its role as a protective species (Abreu et al. (2012) Sci. Transl. Med. 4(151):151ra124; U.S. Pat. No. 10,660,923 B2). Potential mechanisms are via the production of lactic acid thus lowering pH, which commonly implicated pathogenic bacteria such as Pseudomonas do not thrive in, production of antibacterial metabolites, competitive cell surface receptor inhibition reducing pathogenic bacteria adherence, exploitative competition for nutrients starving pathogenic bacteria, promotion of epithelial integrity, and reduction of inflammation. Recent clinical studies in patients with otitis media and recurrent tonsillitis have also demonstrated reduced infections with topical probiotic sprays without significant side effects (Roos et al. (2011) J. Hosp. Infect. 78(1):77-78; Skovbjerg et al. (2008) Arch. Dis. Childhood 94 (2):92-98; Martensson et al. (2022) Clin. Exp. Allergy 52(6):774-783; DeBoeck et al. (2022) Microbiol. Spectr. 10(5):e01682-22). These data are promising with regard to the feasibility/tolerability and effectiveness of topical probiotics in the treatment of disease.

### SUMMARY

Provided herein is a sinus irrigation composition for treating sinusitis in a subject comprising an effective amount of gram-positive Lactobacillales in a pharmaceutically acceptable excipient.

Also provided is a pre-mix composition comprising a gram-positive Lactobacillales and one or more pharmaceutically acceptable auxiliary substances selected from the group consisting of pH adjusting agents/buffering agents, salts, preservatives, surfactants, protectants, humectants, and essential oils.

Further provided is a method of treating sinusitis in a subject comprising introducing an effective amount of a sinus irrigation composition comprising a gram-positive Lactobacillales in a pharmaceutically acceptable excipient into one or more nasal passages of the subject thereby treating sinusitis in the subject.

A kit for the treatment of sinusitis and related conditions is also provided, wherein the kit comprises (i) a pre-mix composition comprising gram-positive Lactobacillales and one or more pharmaceutically acceptable auxiliary substances; (ii) instructions for preparing a sinus irrigation composition by combining a measured volume of water with a measured amount of the pre-mix composition; and (iii) instructions for administering the sinus irrigation composition to a sinus cavity.

### BRIEF DESCRIPTION OF THE DRAWINGS

FIG. 1 shows Trans-Epithelial Resistance (TEER) of human nasal epithelial cell air-liquid interface (HNEC-ALI) cultures after application of L. sakei. Normal saline (0.9% sodium chloride) and L. sakei probiotic at 4.2×10⁶ CFU/mL and 1.5×10⁸ CFU/mL were applied to ALI cultures for 30 minutes. ALI media and 0.5% Triton™ X-100 lysis buffer was used as a negative and positive control, respectively. TEER measurements were taken pre-treatment (t=0), as well as at 5, 10, 30, 60 minutes, and 4 hours after addition of the treatments. Values were normalized to the pre-treatment TEER value. The values are shown as means±SEM for n=3, ***=p<0.001. ANOVA, followed by Dunnett's multiple comparisons post hoc test.

FIG. 2 shows paracellular permeability of HNEC-ALI cultures after application of L. sakei. Passage of fluorescein isothiocyanate (FITC)-dextran recorded 12 hours after a 30-minute treatment period of normal saline (0.9% sodium chloride) and L. sakei (4.2×10⁶ CFU/mL and 1.5×10⁸ CFU/mL) treatments. ALI media and 0.5% Triton™ X-100 were used as a negative and positive control, respectively. The values are shown as means±SEM for n=3. ANOVA, followed by Dunnett's multiple comparisons post hoc test. ***p<0.001.

FIG. 3 shows the percentage of cytotoxic activity from HNEC-ALI cultures after application of L. sakei. Cytotoxicity as determined by the LDH cytotoxicity assay, 4 hours after the addition of normal saline (0.9% sodium chloride) and L. sakei (4.2×10⁶ CFU/mL and 1.5×10⁸ CFU/mL) treatments. ALI media and 0.5% Triton™ X-100 in ALI media was used as a negative and positive control, respectively. Values shown are reported as means±SEM, n=3. ANOVA, followed by Tukey HSD post hoc test, ***p<0.001.

### DETAILED DESCRIPTION OF THE INVENTION

This invention is based, in part, on the finding that formulations including about 4.2×10⁶ CFU/mL to about 1.5×10⁸ CFU/mL Lactobacillus sakei can be used in sinus irrigation applications to treat sinusitis. Accordingly, provided herein are compositions, e.g., a sinus irrigation composition and/or premix composition, containing Lactobacillales bacteria, e.g., L. sakei, that are specifically formulated for sinus irrigation and use of the same in methods of treating sinusitis.

As used herein, a “sinus irrigation composition” refers to an irrigation fluid delivered through the nostril to the nasal passages and/or sinuses of a subject. A sinus irrigation composition may be a liquid, mist, or vapor, e.g., delivered by nebulizers/atomizers.

In some embodiments, the sinus irrigation composition is composed of an effective amount of one or more gram-positive Lactobacillales in a pharmaceutically acceptable excipient. As used herein, a “gram-positive Lactobacillales” refers to a lactic acid bacterium (LAB) falling within the order Lactobacillales. In some embodiments, the lactic acid bacterium used herein meets the following requirements: it adheres to epithelial cells or mucous membranes and is capable of residing therein; it preferably secretes antimicrobial substances to inhibit harmful bacteria, thereby stabilizing the surface it is adhered to and preventing harmful bacteria from residing amongst the epithelial cells or mucous membranes; and it should be safe for use in humans.

In some embodiments, the gram-positive Lactobacillales is a bacterium in a genus selected from the group consisting of Lactobacillus, Leuconostoc, Pediococcus, Lactococcus, Streptococcus, Aerococcus, Carnobacterium, Enterococcus, and Tetragenococcus. In some embodiments, the one or more gram-positive Lactobacillales include bacteria in the genus selected from the group consisting of Lactobacillus (e.g., L. plantarum, L. casei, L. rhamnosus, L. sakei, L. acidophilus, L. gasseri, L. fermentum, L. lactus, L. delbrueckii), Leuconostoc (e.g., L. lactis, L. mesenteroides subsp. cremorii), Pediococcus (e.g., P. pentosaceus), Lactococcus (e.g., L. lactis), and Streptococcus (e.g., S. thermophilus, S. lactis, S. faecalis).

In one embodiment, the compositions and methods herein include the use of one or more Lactobacillus species. In some embodiments, the compositions and methods herein include the use of Lactobacillus sakei. In some embodiments, the probiotic Lactobacillus sakei used herein is available as an over-the-counter supplement sold under the tradename Lanto Sinus™ Probiotic Powder. The probiotic is produced in South Korea using Good Manufacturing Practice standards and the contents of the package (15 g) are advertised as including 17 billion CFU Lactobacillus sakei, and microcrystalline cellulose and dextrose anhydrous as other ingredients. The Lanto Sinus™ Probiotic Powder label specifies mixing 0.25 tsp (about 1 g) Lanto Sinus™ with 0.25 cup (about 59 mL) of distilled or sterile (boiled and cooled) water, i.e., about 1.92×10⁷ CFU/mL. The label directs administration via the nostril by dabbing the mixture about 0.5 inch into the nostrils (using a cotton swab or clean finger) and by mouth by swishing the mixture in the mouth and swallowing. It is recommended that the mixture be applied once in each nostril, once per day, as needed.

In some embodiments, the gram-positive Lactobacillales in the compositions and methods herein is used in combination with a pharmaceutically acceptable excipient or carrier. As used herein “pharmaceutically acceptable excipient” or “pharmaceutically acceptable carrier” includes any and all solvents, dispersion media, coatings, antibacterial and antifungal agents, isotonic agents, and the like that are physiologically compatible with the gram-positive Lactobacillales bacterium (e.g., L. sakei) herein.

In one embodiment, the pharmaceutically acceptable carrier is suitable for nasal administration. Compositions for nasal administration typically include at least one gram-positive Lactobacillales bacterium (e.g., L. sakei), in a pharmaceutically acceptable carrier, e.g., an aqueous carrier. A variety of aqueous carriers may be used, including water, buffered saline, and the like. These solutions are generally free of undesirable matter, e.g., contaminating species.

In some embodiments, a sinus irrigation composition is provided comprising an effective amount of gram-positive Lactobacillales in a pharmaceutically acceptable excipient, wherein the effective amount of gram-positive Lactobacillales (e.g., L. sakei) in the sinus irrigation composition is in the range of about 2.1×10⁴ CFU/mL to about 8.3×10⁹ CFU/mL, e.g., about 2.1×10⁴ CFU/mL, 2.3×10⁴ CFU/mL, 2.5×10⁴ CFU/mL, 2.7×10⁴ CFU/mL, 2.9×10⁴ CFU/mL, 3.1×10⁴ CFU/mL, 3.3×10⁴ CFU/mL, 3.5×10⁴ CFU/mL, 3.7×10⁴ CFU/mL, 3.9×10⁴ CFU/mL, 4.1×10⁴ CFU/mL, 4.3×10⁴ CFU/mL, 4.5×10⁴ CFU/mL to about 7.1×10⁹ CFU/mL, 7.3×10⁹ CFU/mL, 7.5×10⁹ CFU/mL, 7.7×10⁹ CFU/mL, 7.9×10⁹ CFU/mL, 8.1×10⁹ CFU/mL, 8.3×10⁹ CFU/mL, or any value or range therebetween. In some embodiments, a sinus irrigation composition herein includes about 4.2×10⁶ CFU/mL to about 4.2×10⁹ CFU/mL gram-positive Lactobacillales (e.g., L. sakei), e.g., about 4.2×10⁶ CFU/mL, 4.4×10⁶ CFU/mL, 4.6×10⁶ CFU/mL, 4.8×10⁶ CFU/mL, 5.0×10⁶ CFU/mL, 5.2×10⁶ CFU/mL to about 3.2×10⁹ CFU/mL, 3.4×10⁹ CFU/mL, 3.6×10⁹ CFU/mL, 3.8×10⁹ CFU/mL, 4.0×10⁹ CFU/mL, 4.2×10⁹ CFU/mL gram-positive Lactobacillales (e.g., L. sakei). In some embodiments, a sinus irrigation composition herein includes about 8.3×10⁷ CFU/mL of Lactobacillus sakei.

In some embodiments, a sinus irrigation composition herein includes a total of about 1.0×10⁵ CFU to about 5.0×10¹¹ CFU gram-positive Lactobacillales (e.g., L. sakei), e.g., about 5.0×10⁵ CFU, 1.0×10⁶ CFU, 5.0×10⁶ CFU, 1.0×10⁷ CFU, 5.0×10⁷ CFU to about 1.0×10⁹ CFU, 5.0×10⁹ CFU, 1.0×10¹⁰ CFU, 5.0×10¹⁰ CFU, 1.0×10¹¹ CFU, or 5.0×10¹¹, or any range or value therebetween. In some embodiments, a sinus irrigation composition herein includes a total of about 5.0×10⁹ CFU to about 5.0×10¹¹ CFU gram-positive Lactobacillales (e.g., L. sakei), or any range or value therebetween.

In some embodiments, the composition herein is a pre-mix composition comprising a gram-positive Lactobacillales (e.g., a Lactobacillus sp. such as L. sakei) and one or more pharmaceutically acceptable auxiliary substances. A “pre-mix composition” refers to a mixture of ingredients designed to be mixed with other ingredients before use. In some embodiments, the one or more pharmaceutically acceptable auxiliary substances of the pre-mix composition are selected from the group consisting of pH adjusting agents and/or buffering agents, salts, preservatives, surfactants, protectants, humectants, and essential oils. The concentration of active agent (i.e., gram-positive Lactobacillales bacterium such as L. sakei) and auxiliary substances in these formulations may vary widely, and may be selected primarily based on fluid volumes, viscosities, body weight, and the like in accordance with the particular mode of administration selected and the patient's needs.

In one embodiment, a composition herein (e.g., a pre-mix composition) may include one or more pH adjusting agents and/or buffering agents. In certain embodiments, the composition may include an alkaline buffering agent that maintains the pH of the nasal rinse treatment fluid in a range of about 6 to about 9, e.g., about 6, 7, 8, or 9. In some embodiments, the alkaline buffering agent maintains the pH of the nasal rinse treatment fluid in a range of about 7 to about 8, e.g., about 7 or about 8. In other embodiments, the alkaline buffering agent maintains the pH of the nasal rinse treatment fluid in a range of about 7.3 to about 7.5. In other embodiments, the alkaline buffering agent maintains the pH of the nasal rinse treatment fluid at about 7.4. Examples of alkaline buffering agents include, but are not limited to, bicarbonate salts, carbonate salts, sodium or potassium hydroxides, high pH chelating agents, high pH surfactants, and any combination thereof. In some embodiments, the composition may include sodium bicarbonate as the alkaline buffering agent. A person of skill in the art, with the benefit of this disclosure, would be able to select an alkaline buffering agent that may be suitable for a particular application of the present disclosure.

In another embodiment, one or more salts may be present in a composition herein(e.g., a pre-mix composition). Examples of salts suitable for certain embodiments of the present disclosure include, but are not limited to, sodium chloride, calcium chloride, ammonium chloride, magnesium chloride, strontium chloride, barium chloride, potassium chloride, ammonium sulfate, sodium sulfate, magnesium sulfate, aluminum sulfate, sodium bromide, potassium bromide, calcium bromide, zinc bromide, sodium bicarbonate, a carbonate salt, a sulfate salt, a phosphate salt, a magnesium salt, a bromide salt, a formate salt, an acetate salt, a chloride salt, a fluoride salt, a bicarbonate salt, a nitrate salt, a phosphate salt, and any combination thereof. In some embodiments, the composition may include sodium chloride as the salt. The selection of a suitable salt may depend on several factors that would be recognized by a person of skill in the art with the benefit of this disclosure, including but not limited to any other types of salt in the composition of the present disclosure, the characteristics of an aqueous fluid (e.g., pH, salinity, etc.), and the like.

In other embodiments, a composition herein (e.g., a pre-mix composition) may include one or more preservatives. The preservatives may help reduce the growth of microorganisms that may be introduced into the nasal rinse treatment fluid after opening its storage container. Examples of such preservatives include, but are not limited to, grapefruit seed extract, benzalkonium chloride, benzyl alcohol, sodium benzyl chloride, sodium benzoate, potassium sorbate, potassium benzoate, sodium benzyl alcohol, any other preservatives that help reduce the growth of microorganisms, and any combination thereof.

In further embodiments, a composition herein (e.g., a pre-mix composition) may include one or more surfactants. Surfactants may be anionic, cationic, nonionic, or amphoteric/zwitterionic. Examples of anionic surfactants include, by way of non-limiting example, alkyl sulfates, alkyl ethoxylate sulfates, and soaps such as sodium stearate. Non-limiting examples of cationic surfactants include quaternary ammonium salts such as tetradecyl trimethyl ammonium bromide, benzalkonium chloride, and cetylpyridinium chloride. Non-limiting examples of nonionic surfactants that may be used in the compositions herein include polysorbates such as polysorbate 20, Tyloxapol and related 4-(1,1,3,3-tetramethylbutyl) phenol/poly(oxyethylene) polymers, poly(oxyethylene)-poly(oxypropylene) block copolymers, polyethylene glycol esters of fatty acids, such as coconut, polysorbate, polyoxyethylene, polyvinylpyrrolidone (povidone), and polyoxypropylene ethers of higher alkanes (C12-C18). Examples of amphoteric/zwitterionic surfactants include without limitation alkyl betaines. In some embodiments, the surfactant is a quaternary ammonium compound. In some embodiments, the surfactant is benzalkonium chloride. Compounds disclosed herein as surfactants may act as preservatives, and vice versa. Two examples include, e.g., benzalkonium chloride and EDTA.

In some embodiments, a composition herein (e.g., a pre-mix composition) may include one or more mono- and/or disaccharides as protectants in the manufacture of the compositions herein. These protectants may help enhance bacterial survival after spray-drying. Examples of such protectants include, but are not limited to, glucose, mannose, mannitol, dextran, lactose, trehalose, or any other suitable protectant that promotes Lactobacillales survival, and any combination thereof.

In some embodiments, a composition herein (e.g., a pre-mix composition) may include one or more humectants. In some embodiments, the humectant is chosen from xylitol, polyethylene glycol (PEG), glycerin, and mannitol.

In other embodiments, a composition herein (e.g., a pre-mix composition) may include one or more essential oils. Examples of such essential oils include, but are not limited to, tea tree oil, eucalyptus oil, peppermint oil, carrot seed oil, cinnamon oil, rosemary oil, oregano oil, thyme oil, any other essential oils that help reduce gram-positive and gram-negative bacteria growth, and any combination thereof.

In some embodiments, a composition herein (e.g., a sinus irrigation composition or a pre-mix composition) may also include one or more flow agents. Examples of flow agents include sugar alcohols such as xylitol and mannitol. Certain flow agents such as xylitol have antibacterial effects as well.

In some embodiments, the compositions herein (e.g., a sinus irrigation composition or a pre-mix composition) may also include one or more suspending agents/stabilizers. Suspending agents include inorganic materials, synthetic compounds, and polysaccharides. Examples of suspending agents include magnesium stearate.

As is well-known in the art, certain excipients disclosed herein may have multiple functions in a formulation. For example, quaternary ammonium compounds such as benzalkonium chloride can function as a preservative (even in low amounts), a permeation/penetration enhancer, and/or a cationic surfactant (sometimes at a higher amount for these latter two). As another example, cyclodextrins may act as solubility enhancers and/or molecular inclusion agents for slow release of a compound from an inclusion complex.

In addition to one or more gram-positive Lactobacillales bacteria such as L. sakei, a composition herein (e.g., a sinus irrigation composition or a pre-mix composition) may also include additional active compounds, including, e.g., antibiotic or anti-inflammatory agents. Combination therapies contemplate coadministration, using separate formulations or a single pharmaceutical formulation, and consecutive administration in either order.

Compositions such as a sinus irrigation composition or a pre-mix composition may be provided in a container, sachet, or vessel comprising a measured amount of the sinus irrigation composition or the pre-mix composition. As used herein, a “measured amount” refers to any amount needed of a sinus irrigation composition or a pre-mix composition for treating sinusitis or ameliorating at least one sign or symptom of sinusitis. In some embodiments, the term “measured amount” refers to the entire contents of the container, sachet, or vessel. In some embodiments, the measured amount comprises a total of about 1.0×10⁵ CFU to about 5.0×10¹¹ CFU gram-positive Lactobacillales (e.g., L. sakei), e.g., about 5.0×10⁵ CFU, 1.0×10⁶ CFU, 5.0×10⁶ CFU, 1.0×10⁷ CFU, 5.0×10⁷ CFU to about 1.0×10⁹ CFU, 5.0×10⁹ CFU, 1.0×10¹⁰ CFU, 5.0×10¹⁰ CFU, 1.0×10¹¹ CFU, or 5.0×10¹¹, or any range or value therebetween, in combination with a suitable amount of pharmaceutically acceptable auxiliary substance. In some embodiments, the measured amount comprises about 5.0×10⁹ CFU to about 5.0×10¹² CFU gram-positive Lactobacillales (e.g., L. sakei), or any range or value therebetween.

In some embodiments, the pre-mix composition herein is mixed with a suitable aqueous carrier, e.g., water, to provide a sinus irrigation composition of use in the treatment of sinusitis or at least one symptom thereof.

To facilitate use in the treatment of sinusitis, the compositions herein may be provided in the form of a kit. In one embodiment, a kit for the treatment of sinusitis and related conditions is provided, wherein the kit comprises (i) a pre-mix composition comprising a gram-positive Lactobacillales (e.g., a Lactobacillus such as L. sakei) and one or more pharmaceutically acceptable auxiliary substances selected from the group consisting of pH adjusting agents/buffering agents, salts, preservatives, surfactants, protectants, humectants, and essential oils; (ii) instructions for preparing a sinus irrigation composition by combining a measured volume of water (e.g., distilled or boiled water) with a measured amount of the pre-mix composition; and (iii) instructions for administering the sinus irrigation composition to a sinus cavity. In some embodiments, the pre-mix composition is packaged in one or more single use vials, sachets, or vessels containing a pre-measured amount of the pre-mix composition sufficient for a single application of the sinus irrigation composition to a sinus cavity. In some embodiments, the kit may further include (iv) a single or multi-use vessel for combining the pre-mix composition and water; or (v) a single or multi-use vessel or apparatus suitable for sinus irrigation.

In another embodiment, the kit may include (i) a vessel or apparatus suitable for nasal irrigation; (ii) a pre-mix composition as disclosed herein; (iii) instructions for preparing a sinus irrigation composition by combining a measured volume of distilled water with a measured amount of the pre-mix composition in the vessel or apparatus; and (iv) instructions for administering the sinus irrigation composition to a sinus cavity. In an embodiment, the kit comprises one or more single use vials, sachets, or vessels containing a pre-measured amount of the pre-mix composition sufficient for a single application of the nasal irrigation fluid to a sinus cavity.

In another embodiment, the kit may include (i) one or more single-use vessels or apparatus suitable for nasal irrigation, each containing an amount of a pre-mix composition sufficient for a single application of a sinus irrigation composition to a sinus cavity; (ii) instructions for preparing the sinus irrigation composition by combining a measured volume of distilled water with pre-mix composition in the vessel or apparatus; and (iii) instructions for administering the sinus irrigation composition to a sinus cavity.

Advantageously, the compositions and kits herein are of particular use in the treatment, prevention, or amelioration of sinusitis. Accordingly, also provided herein is a method of treating sinusitis in a subject comprising introducing an effective amount of a sinus irrigation composition comprising a gram-positive Lactobacillales (e.g., a Lactobacillus bacterium such as L. sakei) in a pharmaceutically acceptable excipient into one or more nasal passages of the subject thereby treating sinusitis in the subject. In some embodiments, the sinus irrigation composition is prepared by combining a pre-mix composition as described herein with water (e.g., distilled or boiled water).

As used herein, the term “sinusitis” or, equivalent thereof, e.g., “rhinosinusitis,” refers to inflammation of the sinuses, usually caused or exacerbated by infection, and includes acute, recurrent acute, subacute, and chronic sinusitis. Acute sinusitis symptoms last about 2 to 8 weeks. Chronic symptoms last longer, e.g., a few months or a lifetime. Symptoms of sinusitis, and related conditions, include pain and/or pressure, particularly of the facial area and sometimes localized to the affected sinus (es); nasal discharge and post-nasal drip; toothache; headache; fever; and infection of nearby structures such as the eye socket and bone.

Subjects that may be treated by the method herein include any mammal, in particular a human. Human subjects may be of any age and include adults and children. In some embodiments, the subject has sinusitis or an elevated risk of having sinusitis. The following are risk factors for sinusitis and may guide the person of ordinary skill in determining those subjects or patients at risk for developing sinusitis (subject or patient at risk for sinusitis): allergic rhinitis or hay fever; persistent colds; sore throats; asthma; children who regularly attend day care; patients or subjects with diseases that prevent the cilia from working properly, such as Kartagener syndrome and immotile cilia syndrome; patients or subjects who are subjected to changes in altitude (especially activities such as flying or scuba diving); patients or subjects with large adenoids; tooth infections (although somewhat rare); weakened immune system including from HIV, chemotherapy, other. In some embodiments, a subject having an elevated risk of sinusitis is a subject with allergies or asthma. In some embodiments, a subject treated in accordance with the method herein has recalcitrant symptoms of sinusitis and/or recalcitrant sinusitis.

The terms “treat,” “treating,” and “treatment” are not intended to be absolute terms. Treatment can refer to any delay in onset, reduction in the frequency or severity of symptoms, amelioration of symptoms, improvement in patient comfort and/or respiratory function, etc. The effect of treatment may be compared to an individual or pool of individuals not receiving a given treatment, or to the same patient prior to, or after cessation of, treatment.

The term “prevent” refers to a decrease in the occurrence of sinusitis symptoms in a patient. Prevention may be complete (no detectable symptoms) or partial, such that fewer symptoms are observed than would likely occur absent treatment.

In some embodiments, the “effective amount” of a sinus irrigation composition is with reference to the amount of the particular Lactobacillales (e.g., a Lactobacillus bacterium such as L. sakei) strain or species that provides the intended effect of treating or ameliorating sinusitis and/or a symptom thereof. In some embodiments, the effective amount of sinus irrigation composition may be expressed in absolute numbers or ranges of numbers of Lactobacillales (e.g., a Lactobacillus bacterium such as L. sakei), e.g., 10⁵ or 10⁵-10¹¹ Lactobacillales in a composition, or in a given volume. The amount can also be expressed in terms of colony forming units (CFU), or absorbance, depending on the assay used for bacterial detection, as will be appreciated by one of skill. The amount may also be expressed in comparative terms, e.g., compared to a control.

The terms “dose” and “dosage” are used interchangeably herein. A dose refers to the amount of active ingredient given to an individual at each administration. For the present invention, the dose may generally refer to the amount of probiotic, antibiotic, or anti-inflammatory agent. Dosage may also be expressed in terms of bacterial concentration. The dose may vary depending on a number of factors, including frequency of administration; size and tolerance of the individual; severity of the condition; risk of side effects; and the route of administration. A skilled artisan will recognize that the dose may be modified depending on the above factors or based on therapeutic progress.

In some embodiments, an effective amount of a sinus irrigation composition may be a daily dose in the range of about 1.0×10⁵ CFU to about 5.0×10¹² CFU gram-positive Lactobacillales (e.g., L. sakei), e.g., about 5.0×10⁵ CFU, 1.0×10⁶ CFU, 5.0×10⁶ CFU, 1.0×10⁷ CFU, 5.0×10⁷ CFU to about 1.0×10⁹ CFU, 5.0×10⁹ CFU, 1.0×10¹⁰ CFU, 5.0×10¹⁰ CFU, 1.0×10¹¹ CFU, 5.0×10¹¹, 1.0×10¹² CFU, or 5.0×10¹² per day, or any range or value therebetween. In some embodiments, an effective amount of a sinus irrigation composition may be a dose in the range of about 1.0×10⁷ CFU to about 4.0×10¹² CFU gram-positive Lactobacillales (e.g., L. sakei) per day, or any range or value therebetween. In some embodiments, an effective amount of a sinus irrigation composition may be a dose in the range of about 2.0×10⁹ CFU to about 2.0×10¹² CFU gram-positive Lactobacillales (e.g., L. sakei) per day, or any range or value therebetween. In some embodiments, an effective amount of a sinus irrigation composition may be a dose in the range of about 3.0×10¹⁰ CFU to about 5.0×10¹⁰ CFU gram-positive Lactobacillales (e.g., L. sakei) per day, or any range or value therebetween.

In some embodiments, the effective amount of sinus irrigation composition is administered in a volume in the range of at least about 50 mL, e.g., at least about 50 mL, 60 mL, 70 mL, 80 mL, 90 mL, 100 mL, 110 mL, 120 mL, 130 mL, 140 mL, 150 mL, 160 mL, 170 mL, 180 mL, 190 mL, 200 mL, 210 mL, 220 mL, 230 mL, 240 mL, 250 mL, 260 mL, 270 mL, 280 mL, 290 mL, 300 mL, 310 mL, 320 mL, 330 mL, 340 mL, 350 mL, 360 mL, 370 mL, 380 mL, 390 mL, 400 mL, 410 mL, 420 mL, 430 mL, 440 mL, 450 mL, 460 mL, 470 mL, 480 mL, 490 mL, or 500 mL. In some embodiments, a sinus irrigation composition comprises a volume of at least about 100 mL to about 500 mL, or any range or value therebetween. In some embodiments, a sinus irrigation composition comprises a volume of at least about 240 mL.

In some embodiments, an effective amount of the sinus irrigation composition is the amount administered to each nasal passage. In some embodiments, an effective amount of the sinus irrigation composition is the total amount administered to both nasal passages.

In some embodiments, the effective amount of the sinus irrigation composition is administered on a daily basis (e.g., once per day) for one or more days, e.g., one, two, three, four, five, six, seven, eight, nine, 10, 11, 12, 13, 14, or 15 days. In some embodiments, the effective amount of the sinus irrigation composition is divided into two or more daily doses, e.g., one, two, three, or four daily doses, and administered to the subject for one or more days, e.g., one, two, three, four, five, six, seven, eight, nine, 10, 11, 12, 13, 14, or 15 days. By way of illustration, an effective amount of a sinus irrigation composition may be 4×101° C. FU Lactobacillales (e.g., L. sakei) per day that is divided into two doses, wherein the first daily dose of 2×10¹⁰ CFU is administered in the morning (e.g., 1×10¹⁰ CFU per nasal passage) and the second daily dose of 2×10¹⁰ CFU is administered in the evening (e.g., 1×10¹⁰ CFU per nasal passage).

An effective amount may be determined by practices readily carried out by the skilled clinician. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions may contain such amounts or submultiples thereof to make up the daily dose. The dosage may be adjusted by the individual physician in the event of any contraindications. It is generally preferred that a maximum dose of a therapeutic agent (alone or combination with other therapeutic agents) be used, that is, the highest safe dose according to sound medical judgment. It will be understood by those of ordinary skill in the art however, that a patient may insist upon a lower dose or tolerable dose for medical reasons psychological reasons or for virtually any other reasons.

Among the many potential advantages to the methods and compositions herein, only some of which are alluded to herein, the methods and compositions of the present disclosure may provide for reduced inflammation of one or more nasal passages. For example, in some embodiments, the methods and compositions of the present disclosure may reduce nasal infection, rhinitis, or sinusitis when the sinus irrigation composition is administered in one or more nasal passages. Accordingly, the methods and compositions of the present disclosure may reduce facial pressure and pain that results from the impairment of sinus function and drainage. Moreover, in some embodiments, the methods and compositions of the present disclosure may provide a nasal rinse treatment fluid that is a mucolytic agent that will reduce mucus viscosity and promote mucociliary clearance in one or more nasal passages. Furthermore, the methods and compositions of the present disclosure may enhance respiratory functions, particularly as compared with the respiratory conditions associated with allergic rhinitis, acute rhinitis, chronic rhinitis, allergic sinusitis, acute sinusitis, chronic sinusitis, cystic fibrosis, and any other conditions marked by excess mucus.

Also provided herein are nasal irrigation delivery devices comprising the sinus irrigation composition or pre-mix composition disclosed herein. Nasal irrigation delivery devices suitable for use may be single-use or multiple-use.

A single-use product may be sterile prior to opening of the package and all the contents in the package are intended to be used in a single application to one or both nasal passages of a subject. The single-dose packaging arrangement may be made by any known method. Examples of such packaging methods include blow-filled seals and conventional plastic bottles.

The compositions of the invention may be administered using an applicator or delivery device adapted for the administration of sinus irrigation compositions to the nasal passages. In some embodiments, the applicator or delivery device includes a structure thereon that is adapted for nasal delivery of a sinus irrigation composition. According to some embodiments, the delivery device for the sinus irrigation composition is a positive-pressure squeeze device. Examples of squeeze devices that may be used with the sinus irrigation composition herein include, without limitation, bottles and bulb syringes.

According to some embodiments, delivery of the sinus irrigation composition may use a delivery device to deliver a pressurized stream of the sinus irrigation composition to the nasal passage of a subject. Examples of delivery devices and applicators suitable for use with the compositions herein may be found, for example, in U.S. Pat. No. 5,806,723 (“Device for Lavaging”), U.S. Pat. No. 3,847,145 (“Nasal Irrigation System”), U.S. Pat. No. 6,520,384 (“Apparatus and Method for Nasal Rinse”), U.S. Pat. No. 6,669,059 (“System and Method for Passage Rinse”), U.S. Pat. No. 6,540,718 (“Appliance for Rinsing”), WO 2011/094504 (“Antimicrobial Sinus Irrigation Compositions, Methods, and Devices”), and U.S. Patent Publication No. 2014/0371305 (“Mupirocin Antibiotic Composition”).

According to some embodiments, the delivery device may be a plastic squeeze bottle that creates a seal to the nostril in order to deliver a stream of a sinus irrigation composition into the sinus passages. Commercially available examples of suitable nasal irrigation devices include those similar to the Micfit 250 mL SinuClear Squeeze Bottle; the 8 oz NasoPure® squeeze bottle; the SinuCleanse® Squeeze Nasal Wash Bottle, the ActiveSinus Sinus Rinse Bottle, or the NeilMed® Sinus Rinse Bottle. Children's nasal irrigation devices, which may deliver a smaller volume of fluid, are also contemplated. In some embodiments, the device delivers the sinus irrigation composition with sufficient pressure to reach and/or coat the sinuses, and in some embodiments, the deep sinuses specifically; this would be more pressure, for example, than the relatively neutral pressure due to gravity that a neti pot would deliver.

In some embodiments, the device for delivery of the sinus irrigation composition may be an active sinus device. For example, an electric nasal irrigation device may be used. Such devices are known in the art, and typically comprise a reservoir for holding fluid, a mechanism for delivering fluid, often driven by an electric motor, and at least one nosepiece that conforms to the shape of the nostril. Examples include devices which deliver fluid to the sinuses by gentle ejection, such as the Sinupulse and Hydro Pulse devices as well as the cordless, handheld Sinugator device from NeilMed, and devices which use gentle suction, delivered through two nosepieces, to draw fluid through the sinus passages, such as the Naväge device.

The device for delivery of a sinus irrigation composition may also be a sinus atomizer or nebulizer. Such devices may deliver a mist containing active ingredient to the sinuses. Examples of such devices include those made by Nebology, such as the PARI SINUS Pulsating Aerosol System. Additional suitable delivery devices are disclosed in Albu (2012) Drug Design, Development and Therapy 6:125-132.

The foregoing may be better understood by reference to the following examples, which are presented for purposes of illustration and are not intended to limit the scope of the invention.

### Example 1: Materials and Methods

Probiotic Powder. Lactobacillus sakei probiotic powder was obtained from Lanto Health (Lanto Health, NJ, USA). L. sakei probiotic suspension was prepared using physiologic saline (0.9% (w/v) NaCl) to 0.5 McFarland standard optical density (OD) at 600 nm using a DensiCHEK_Plus spectrophotometer (Biomerieux), which corresponds to 1.5×10⁸ colony forming units (CFU) per milliliter. This suspension was then further diluted with saline to desired probiotic L. sakei concentrations used in this study.

Primary Human Nasal Epithelial Cell Culture. Nasal brushings were obtained from voluntary individuals undergoing endoscopic sinus surgery after written informed consent. Individuals were excluded if they were less than 18 years of age. Nasal brushings were collected from the middle and inferior turbinate using sterile cytology brushes (CooperSurgical, CT) and transported in supplemented Dulbecco's Modified Eagle Medium/Nutrient Mixture F-12 (DMEM/F-12) (Thermo Fisher Scientific Inc, MA) on ice. Sinonasal epithelial cells underwent centrifugation (150×g for 3 minutes) after being released from brushes. Excess media was aspirated, and the cell pellet was carefully resuspended in supplemented Airway Epithelial Cell Growth Medium (PromoCell, Heidelberg, Germany) with 10 μM of Y-27632 (Sigma-Aldrich Inc, MO) and seeded onto a 50 μg/mL rat tail collagen type 1 coated 100 mm cell culture dish (Thermo Fisher Scientific Inc, MA). After reaching 80% confluence, 0.25% trypsin was used to harvest cells for seeding onto a rat tail type 1 collagen coated 6.5 mm permeable Transwell® insert with 0.4 μm pores at a density of 5×10⁴ cells per insert. Cells were again maintained in supplemented Airway Epithelial Cell Growth Medium with 10 μM of Y-27632 in both the apical and basolateral spaces for 3-4 days or until confluent at 37° C. with 5% CO₂. The apical media was carefully aspirated, and the basal media was replaced with PneumaCult®-ALI Basal Medium (STEMCELL Technologies Inc, Vancouver, Canada) (ALI media), beginning the differentiation process. During the differentiation phase, the apical surface of the wells was also washed with PBS to remove excess mucus every 3 days. Human nasal epithelial cultures at air-liquid interface (ALI) were maintained for at least 28 days for fully differentiated cultures.

Transepithelial Electrical Resistance (TEER). Transepithelial electrical resistance was evaluated using an EVOM2 epithelial Volt/Ohm meter (World Precision Instruments, FL), and results are expressed as a relative change from the initial readings. Air-liquid interface cultures were used only with TEER values of >400 Ω/cm². At the time of measurement, 200 μL of ALI media was added to the apical insert in order to create an electric current between the apical and basal wells. A TEER measurement was recorded as time=0 for each well prior to application of treatments. Apical media was then aspirated and replaced with 100 μL of normal saline (0.9% sodium chloride), L. sakei in normal saline at indicated concentrations, ALI media (negative control), and 0.5% Triton™ X-100 (positive control). Treatments were applied for 30 minutes. After incubation period, treatments were aspirated and cells were washed with ALI media followed by TEER measurements at indicated time points (5, 10, 30, 60 minutes, and 4 hours).

Permeability Assay. The paracellular permeability of the human nasal epithelial cell ALI cultures was evaluated after 30 minutes treatment exposure. After exposure period, treatments in the apical well were aspirated, washed, and incubated with ALI media in the basolateral well for 4 hours. Four kDa fluorescein isothiocyanate (FITC) dextran (Sigma-Aldrich, St. Louis, MO) was applied to the apical well for 12 hours to a concentration of 2 mg/mL. Passage of FITC-dextran into the basolateral compartment was measured by a BioTek Synergy 2 microplate reader (BioTek Instruments, Inc, Vermont) using wavelengths 485 nm and 520 nm for excitation and emission, respectively.

Cytotoxicity Assay. Cytotoxicity from 30 minutes of treatment exposure on human nasal epithelial cell ALI cultures was evaluated through lactate dehydrogenase (LDH) assay. After the exposure period, the apical well was aspirated, washed with ALI media, and incubated for 4 hours at 37° C. with 5% CO₂. LDH was measured using LDH Cytotoxicity Assay (Thermo Fisher Scientific Inc, MA). Briefly, 50 μL from each culture was transferred to a 96-well plate, and 50 μL of LDH reagent was added to the supernatant and incubated for 30 minutes in the dark at room temperature. The absorbance of each well was measured at 680 nm and 490 nm on a BioTek Synergy 2 microplate reader (BioTek Instruments, Inc, Vermont). The percent cytotoxicity was calculated according to the manufacturer's instructions.

Immunofluorescence Microscopy. ALI cultures were washed repeatedly with PBS followed by adding ice-cold (−20° C.) methanol in the apical and basal wells and incubating the plate overnight at −20° C. The following day, methanol was aspirated, and the bottom portion of the ALI membrane was dipped into −20° C. ice-cold acetone for 1 minute. The inserts were then set upside down to air-dry. Blocking buffer (10% normal goat serum, 10% normal bovine serum, 1% bovine serum albumin, 0.01% sodium azide, PBS) was added to the apical and basal wells for 1 hour at room temperature. Mouse monoclonal ZO-1 (Invitrogen, Carlsbad, CA) was diluted 1:300 in blocking solution and incubated at 4° C. overnight. Primary antibody was aspirated and then DyLight® 488 secondary polyclonal anti-mouse IgG antibody (GeneTex, CA) was diluted 1:500 in blocking solution and incubated in the dark for 1 hour at room temperature. Membranes were washed with PBS and a 1 mg/mL solution of 4′,6-diamidino-2-phenylindole (DAPI; Sigma Aldrich) was diluted 1:1000 in blocking solution and membranes were stained for 10 minutes in order to visualize nuclei. After the incubation period, the membrane was washed with PBS and subsequently removed from plastic insert and transferred to a glass slide and a drop of Fluoromount-G® mounting medium was added before cover-slipping. Samples were visualized with EVOSM5000 microscope (Thermo Fisher Scientific Inc, MA).

Statistical Analysis. Data was handled and processed in Microsoft Excel 2013 and graphed in Graphpad Prism v8.4.3 (Dotmatics, CA). All data shown as mean±SEM. All analysis was performed in Graphpad using ANOVA, with either Tukey HSD or Dunnett's multiple comparisons test as indicated, where p values <0.05 were considered statistically significant.

Clinical Testing of Lactobacillus sakei Sinus Irrigation Formulation. A prospective, double blinded, and randomized controlled trial is conducted to assess whether or not topical probiotic sinus irrigations improve symptom severity, sinus microbiota composition, and endoscopy findings, compared to saline sinus irrigations in patients with recalcitrant CRS.

More particularly, the study will demonstrate that topical probiotic irrigations can improve quality-of life measures (Sinonasal Outcome Test-22 [SNOT-22] symptom severity score) compared to saline irrigations; topical probiotic irrigations can improve sinus microbiota (bacterial burden and number of taxa) compared to saline irrigations; and topical antibiotic irrigations can improve endoscopy findings (Lund-Kennedy endoscopic score) compared to saline irrigations.

Sinonasal Outcome Test-22 (SNOT-22). SNOT-22 is a 22 item, validated questionnaire which measures rhinosinusitis specific quality of life outcomes (Hopkins et al. (2009) Clin. Otolaryngol. 34:447-454).

Sinus Microbiota. Nasal swab samples from the study participants are assessed for the number of bacteria present in their nasal cavity before and after treatment. Briefly, bacterial DNA are extracted from nasal swabs according to the manufacturer's instructions (QIAgen). The purified DNA is quantified for DNA concentration using a Qubit fluorometer (Invitrogen, CA) before storing at −20° C. until further use. Bacterial burden is determined by quantitative PCR (qPCR) using universal 16s ribosomal RNA primers. The 331F/797R primers are used for the quantification of total bacteria, as the primers target the V3-V4 hypervariable regions as in Illumina MiSeq. qPCR is carried out using a QuantStudio 3 real-time PCR system (Applied biosystems). All qPCR assays are performed in triplicate. Precautions are taken to ensure that the data from each triplicate falls within 0.5 threshold cycle (Ct), and clear outliers (>2 standard deviations) are removed before calculating average Ct of each sample.

Next, each nasal swab sample is assessed for its bacteria taxonomic distribution by next generation sequencing (NGS) using the MiSeq NGS platform (Illumina) before and after treatment. Briefly, MiSeq paired-end sequencing of the hypervariable V3-V4 regions of the 16 rRNA gene (primers 341F/785R) is performed according to the manual from Illumina. The pooled libraries are sequenced using paired end 2×300 bp reads and a MiSeq v3 reagent kit with 5% PhiX as spike-in. Processing and quality control of the reads are performed for each run separately using R software. This entails quality filtering of the reads, dereplication, denoising, removal of chimeras, and read classification. Taxonomic classification of 16S rRNA targeted amplicon reads is done using the Illumina 16S Metagenomics software. Data handling and visualization is also performed in R, with statistical significance analysis and graphs done using GraphPad Prism.

Lund-Kennedy Endoscopic Score. Endoscopic appearance is collected and scored according to the known the Lund-Kennedy endoscopic score (Lund & Kennedy (1997) Otolaryngol Head Neck Surg. 117(3 Pt 2):535-40).

Visual Analog Scale (VAS) for Pain Associated with Irrigations. The VAS is a continuous scale comprised of a 100-millimeter horizontal line, bounded by no pain (left end) and worst imaginable pain (right end). Respondents mark a location on the line corresponding to the amount of pain experienced, the response is recorded as #millimeters from the left end of line.

Study Design. Subjects are enrolled into each of two study groups: (1) SAL group, wherein subjects receive standard of care topical saline sinus irrigations, and (2) LAC group, where subjects receive topical probiotic irrigations with Lactobacillus sakei. Each subject remains on the study for approximately 6 weeks. There are a total of 3 in-person study visits and one additional telephone contact visit. Prospective and retrospective data collection begins the day of screening and enrollment (Visit 1).

Product Preparation. Product preparation and packaging for the two study groups are carried out by Investigational Drug Service (IDS) in such a manner that neither study personnel nor study patients know which study product they are taking during the study. Both study personnel and study patients are blinded as to group assignment. IDS runs a randomization for the patients to be included in the study and follows the randomization list as patients are enrolled. Once IDS is notified, they prepare the 28 capsules for the patient (2 capsules to be used per day for 14 days) according to the randomized group assignment. IDS keeps the log identifying patient name to the treatment received. Only IDS knows what product each patient received. Study personnel pick up the blinded study capsules from IDS and provide them to the patient for study use. Patients are asked to complete the product use log throughout the course of study involvement. IDS will also provide each subject with bottles of sterile water for irrigation. The study team will provide each subject with a 240 mL sinus rinse bottle. Each sinus rinse bottle will be circumferentially covered with an opaque shrink wrap to obscure any differences in color and texture between the SAL and the LAC solution. This wrap will stop at the water fill line.

Visit 1. The initial visit includes: (1) eligibility screening, (2) signing of the study consent for individuals that meet all study inclusion criteria, (3) double blinded assignment of patients by IDS into one of the 2 study groups, and (4) data collection including demographics (age, gender, race), name, medical record number, phone number and email address, medical history specifically relating to CRS, diagnosis, CT scan assessment using Lund-MacKay CT staging system to assess disease severity, CRS predictors present or absent (nasal polyposis, asthma, aspirin sensitivity, smoking, inhalant allergies, depression), sinus surgery notes, time of surgery, procedure (s) performed, surgical findings, previous treatments, and treatment results. In addition, subjects are asked to complete the 22 item SNOT-22 questionnaire and VAS Scale. Moreover, subjects are provided instructions on how to prepare the sinus irrigation solution. Independent of group assignment, each study subject will irrigate with the appropriate solution 2 times per day for 14 days. Patients begin the irrigations the day after their initial visit. A telephone contact visit will also be conducted one week after initiation of the study intervention. At this telephone visit, subjects will be asked if they have any issues/questions related to the study. Written notes will be taken of the conversation. No additional data will be collected.

Follow-up Visits. Subjects have a total of 3 total study visits, which coincide with standard of care visits for patients being treated for CRS. After the initial enrollment visit (visit 1), follow-up visits occur 3 weeks after beginning sinus irrigations and again at 6 weeks after beginning sinus irrigations. At each follow-up visit, subjects undergo standard of care endoscopic evaluation and complete both the SNOT-22 questionnaire and the VAS Scale.

All scheduled visits, evaluations performed, and data gathered as described above are a part of standard of care for the investigators' CRS patients. The research related activities include explanation of the study to potential participants, patient consent for the study and the comparison between SAL and LAC for the treatment of CRS.

Treatment Groups-Irrigation Preparation. Subjects in both groups will be provided an individual capsule for each scheduled sinus irrigation. Additionally, subjects in both groups will be provided instructions on sinus irrigation product preparation as well as a 240 ml NeilMed® sinus rinse bottle and sterile water. Sinus irrigation is a common procedure for patients with CRS. Training as necessary will be provided. The information sheet provided to subjects will instruct them to: 1) wash their hands before preparing the rinse and before and after each irrigation, 2) ensure sterile water is lukewarm before use, and 3) how to properly clean their sinus rinse bottle and its components after each use.

Sinus Irrigation Solution Preparation. Patients are instructed to (1) empty the contents of one study product capsule into the 240 ml NeilMed® sinus rinse bottle and then (2) add 240 mL of lukewarm distilled water or 240 ml of previously boiled water into the 240 mL NeilMed® sinus rinse bottle. If not using distilled water, the tap water must be boiled before preparing the nose rinse to alleviate the chance of infection. The day after Visit 1, patients in both groups begin the irrigation procedure at home twice a day for 14 days.

The irrigation procedure is performed over a sink. The patient leans forward and slightly turns their head to the left side. The tip of the irrigation bottle is placed into the right nostril and the bottle squeezed using half of the contents of the bottle. The fluid expressed in the right nostril drains out of the left nostril. The participant then lifts their head and blows their nose. The same procedure is completed on the opposite nostril by gently leaning over the sink, tipping the head slightly to the right, placing the tip of the NeilMed® sinus bottle into the left nostril and squeezing the bottle to express the remaining solution into the left sinus area. The solution drains out of the right nostril. Once all the solution has been used, the participant straightens their head and blows their nose. This procedure is performed 2 times/day for 14 days (Table 1).

TABLE 1

| | Per day dose | Total dose |
|---|---|---|
| Per treatment dose | (Treatments twice daily) | (for 14 days) |
| 2 × 10¹⁰ CFU L. | 4 × 10¹⁰ CFU per day of | 10.36 g Lanto |
| Sakei in 240 ml | Lanto Sinus ® Probiotic | Sinus ® |
| water | Powder for two | Probiotic |
| [8.3 × 10⁷ CFU/ml] = | treatments. | Powder |
| 0.37 g Lanto | 0.74 g Lanto Sinus® | |
| Sinus ® Probiotic | Probiotic Powder per | |
| Powder per | day for two treatments | |
| treatment | | |

Patients are asked to return to the ENT clinic 2 times after completing the at home irrigation procedures. The two visits are considered standard of care for patients with active CRS. Patients are asked to return 3 weeks and 6 weeks after beginning the home irrigation process. At each follow-up visit patients undergo standard of care assessments including endoscopic evaluation (Lund-Kennedy score), nasal culture swabbing, SNOT-22 questionnaire, and VAS to measure nasal/sinus pain.

Except for the consent and enrollment procedures, and the use of the probiotic being used as a comparison to the standard of care saline nasal rinses used for CRS treatment, all other activities, visits, and procedures conducted during the study are standard of care. Saline irrigations are a standard treatment for patients with CRS. Irrigations with Lactobacillus sakei are not considered standard of care and are being done for research purposes.

### Example 2: Effect of Topical Probiotic L. sakei on Trans-Epithelial Resistance (TEER) of HNEC-ALI Cultures

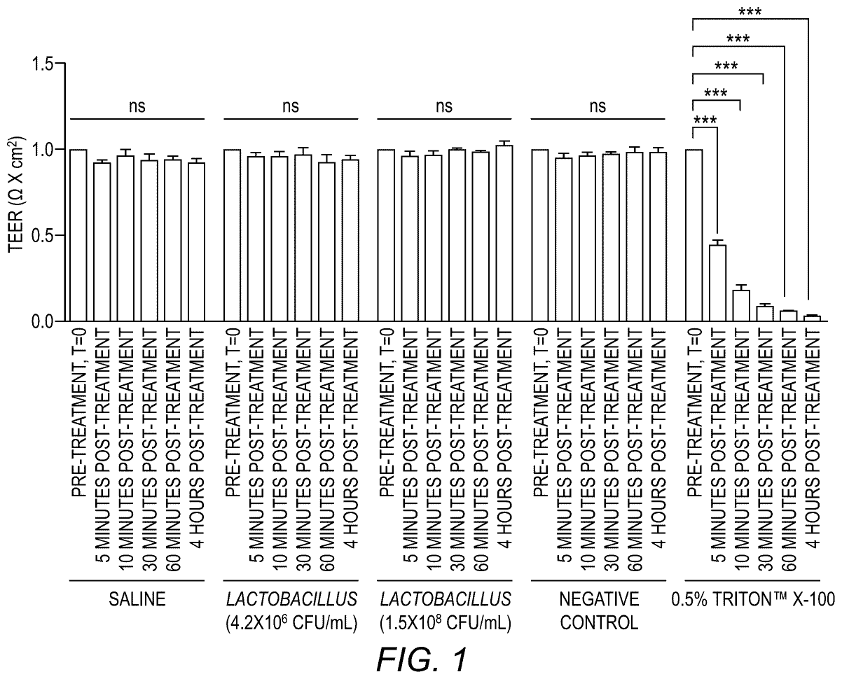

Human nasal epithelial cells (HNEC) were differentiated at air-liquid interface (ALI) derived from 3 independent donors. After 30 minutes of exposure, topical probiotic L. sakei at 4.2×10⁶ CFU/mL and 1.5×10⁶ CFU/mL did not show any significant reduction in TEER at 5 minutes, 10 minutes, 30 minutes, 60 minutes, and 4 hours (p>0.05) (FIG. 1). Similarly, normal saline, and the negative control (ALI media) did not show any significant decreases in TEER at 5 minutes, 10 minutes, 30 minutes, 60 minutes, and 4 hours (p>0.05) (FIG. 1). The positive control, 0.5% Triton™ X-100, showed a significant decline in TEER at all time points after 30 minutes of exposure (FIG. 1) (p<0.05).

### Example 3: Effect of Topical Probiotic L. sakei on the Paracellular Permeability of FITC-Dextran of HNEC-ALI Cultures

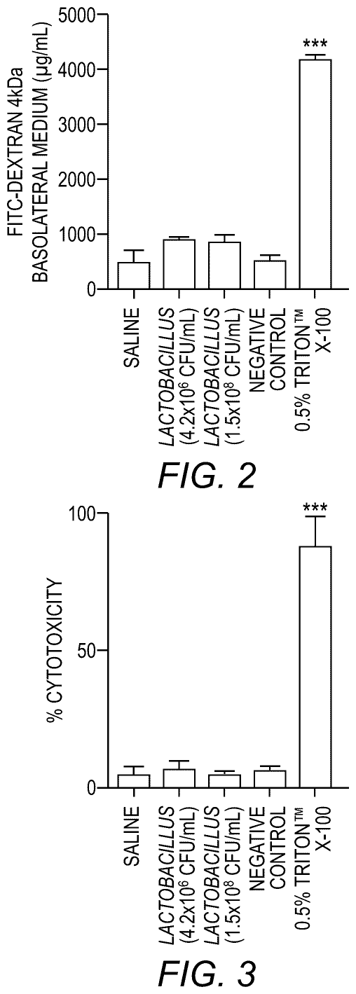

Exposure of topical probiotic L. sakei at 4.2×10⁶ CFU/mL and 1.5×10⁸ CFU/mL for 30 minutes did not show any significant changes to paracellular permeability in HNEC compared to the negative control, ALI media, measured 12 hours after addition of treatments (p>0.05). Normal saline treatment for 30 minutes also revealed no significant changes to paracellular permeability compared to control (p>0.05). The positive control, 0.5% Triton™ X-100, revealed a significant effect on paracellular permeability of HNECs after 30 minutes of exposure (p<0.05) (FIG. 2).

### Example 4: Effect of Topical Probiotic L. sakei on the Immunolocalization of the Tight Junction Protein Zona Occludens-1 (ZO-1) in HNEC-ALI Cultures

The result of 30-minute treatments on the immunolocalization of tight-junction protein, ZO-1, was assessed through immunofluorescence. In both treated and untreated cells, the ZO-1 protein was detected at the periphery of apical cells, as anticipated. No visible disruption of immunolocalization of ZO-1 protein was observed for either topical probiotic L. sakei concentration. The positive control, 0.5% Triton™ X-100, showed significant disruption of ZO-1.

### Example 5: Effect of Topical Probiotic L. sakei on the Lactate Dehydrogenase (LDH) Release from HNECALI Cultures

Percent cytotoxicity was assessed through a LDH release assay from HNECs 4 hours after 30-minute application of topical probiotic L. sakei at concentrations of 4.2×10⁶ CFU/mL and 1.5×10⁸ CFU/mL, ALI media, and 0.5% Triton™ X-100. Topical probiotic L. sakei at concentrations of 4.2×10⁶ CFU/mL and 1.5×10⁸ CFU/mL did not significantly alter paracellular permeability (p>0.05). Normal saline and ALI media also revealed no significant increase in LDH release (p>0.05). The positive control, 0.5% Triton™ X-100, revealed significant release of LDH HNECs after 30 minutes of exposure (p<0.001) (FIG. 3).

### Example 6: Discussion

Air-liquid interface cultures are the gold standard in vitro human airway cell culture system that effectively proliferates and differentiates into a pseudostratified airway epithelium, complete with motile cilia and mucus production and comparable to the in vivo human airway epithelium. They have been used for decades in a variety of research applications, including airway toxicity studies. While immortalized respiratory cell lines such as BEAS-2B, 16HBE1400, Calu-3, A549, NCI-H441 are commercially available for toxicity studies, this study used primary human nasal epithelial cells cultured at air-liquid interface due to the superior capability of these cells to reflect the physiologic characteristics of the human sinonasal airway compared to commercial cell lines.

In a murine model of sinusitis, it has been shown that intranasal L. sakei probiotic (2.5×10⁹ CFU of L. sakei total per treatment) affords protection against intranasal challenge of pathogenic Corynebacterium tuberculostearicum (Abreu et al. (2012) Sci. Transl. Med. 4(151):151ra124). As described herein, the safety profile of 1×10⁹ CFU L. sakei in 240 ml water and 4.2×10⁶ CFU L. sakei/ml were analyzed on differentiated HNEC. In addition, a maximum exposure period of 30 minutes was chosen to evaluate different mucosal barrier function tests and toxicity studies using the in vitro cell culture system.

This analysis indicated that differentiated HNEC exposed to 30 minutes of topical L. sakei at 4.2×10⁶ CFU CFU/mL and 1.5×10⁸ CFU/mL did not show any significant alteration to TEER for up to four hours past exposure period. The positive control, 0.5% of Triton™ X-100 lysis buffer, demonstrated significant reduction to TEER that progressively declined after 5 minutes and persisted up to 4 hours. Similar to the TEER response, the paracellular permeability assays, as measured by the passage of FITC-dextrans into the basolateral well, demonstrated no significant increase to membrane permeability after 30-minute application of L. sakei at either concentration. Furthermore, L. sakei did not disrupt the immunolocalization of tight-junction protein, ZO-1. Taken together, these results demonstrate that 30-minute exposure of L. sakei at these two concentrations do not have detrimental effects on the pore pathway or leak pathway or the immunolocalization of ZO-1 compared to control.

Cytotoxicity, as determined by measuring LDH release after cells have been challenged by a compound, is a well-recognized in vitro technique for toxicity assessment. The LDH measured from the differentiated HNECs after 30-minute exposure to L. sakei was not significantly different than the negative control (ALI Basal media) or with sodium chloride, the diluent used for L. sakei.

These results demonstrate a good safety profile of L. sakei at the indicated concentrations when applied to nasal epithelial cells in vitro and indicate the safety of using topical L. sakei at the tested concentrations in individuals with CRS to restore the sinonasal microbiome.

The detailed description set forth above is provided to aid those skilled in the art in practicing the present invention. However, the invention described and claimed herein is not to be limited in scope by the specific embodiments herein disclosed because these embodiments are intended as illustration of several aspects of the disclosure. Any equivalent embodiments are intended to be within the scope of the invention. Indeed, various modifications of the disclosure in addition to those shown and described herein will become apparent to those skilled in the art from the foregoing description, which do not depart from the spirit or scope of the present invention. Such modifications are also intended to fall within the scope of the appended claims.

What is claimed is:

1. A sinus irrigation composition for treating sinusitis in a subject comprising an effective amount of gram-positive Lactobacillales in a pharmaceutically acceptable excipient.

2. The sinus irrigation composition of claim 1, comprising about 2.1×10⁴ colony forming units (CFU)/mL to about 8.3×10⁹ CFU/mL of the gram-positive Lactobacillales in a pharmaceutically acceptable excipient.

3. The sinus irrigation composition of claim 1, wherein the Lactobacillales is selected from the group consisting of Lactobacillus, Leuconostoc, Pediococcus, Lactococcus, Streptococcus, Aerococcus, Carnobacterium, Enterococcus, and Tetragenococcus.

4. The sinus irrigation composition of claim 1, wherein the Lactobacillales is Lactobacillus sakei.

5. The sinus irrigation composition of claim 4, comprising about 4.2×10⁶ colony forming units (CFU)/mL to about 4.2×10⁹ CFU/mL of Lactobacillus sakei in a pharmaceutically acceptable excipient.

6. The sinus irrigation composition of claim 4, comprising about 8.3×10⁷ colony forming units (CFU)/mL of Lactobacillus sakei in a pharmaceutically acceptable excipient.

7. A pre-mix composition comprising a gram-positive Lactobacillales and one or more pharmaceutically acceptable auxiliary substances selected from the group consisting of pH adjusting agents, buffering agents, salts, preservatives, surfactants, protectants, humectants, and essential oils.

8. A container, sachet, or vessel comprising a measured amount of the pre-mix composition of claim 7.

9. A method of treating sinusitis in a subject comprising introducing an effective amount of a sinus irrigation composition comprising a gram-positive Lactobacillales in a pharmaceutically acceptable excipient into one or more nasal passages of the subject thereby treating sinusitis in the subject.

10. The method of claim 9, wherein the effective amount of the gram-positive Lactobacillales composition is administered on a daily basis for one or more days.

11. The method of claim 10, wherein the effective amount the gram-positive Lactobacillales composition is divided into two or more dosages for administration to the subject.

12. The method of claim 9, comprising administering about 1×10⁷ colony forming units (CFU) to about 4×10¹² CFU per day of the gram-positive Lactobacillales.

13. The method of claim 12, wherein the Lactobacillales is selected from the group consisting of Lactobacillus, Leuconostoc, Pediococcus, Lactococcus, Streptococcus, Aerococcus, Carnobacterium, Enterococcus, and Tetragenococcus.

14. The method of claim 13, wherein the Lactobacillales is Lactobacillus sakei.

15. The method of claim 14, wherein the effective amount of Lactobacillus sakei is administered to the subject in two or more dosages daily.

16. The method of claim 14, comprising administering about 2×10⁹ colony forming units (CFU) to about 2×10¹² CFU per day of Lactobacillus sakei.

17. The method of claim 14, comprising administering about 3×101⁰ colony forming units (CFU) to about 5×10¹⁰ CFU per day of Lactobacillus sakei.

18. A kit for the treatment of sinusitis and related conditions comprising
(i) a pre-mix composition comprising a gram-positive Lactobacillales and one or more pharmaceutically acceptable auxiliary substances selected from the group consisting of pH adjusting agents/buffering agents, salts, preservatives, surfactants, protectants, humectants, and essential oils;
(ii) instructions for preparing a sinus irrigation composition by combining a measured volume of water with a measured amount of the pre-mix composition; and
(iii) instructions for administering the sinus irrigation composition to a sinus cavity.

19. The kit of claim 18, wherein the pre-mix composition is packaged in one or more single use vials, sachets, or vessels containing a pre-measured amount of the pre-mix composition sufficient for a single application of the sinus irrigation composition to a sinus cavity.

20. The kit of claim 18, further comprising
(iv) a single or multi-use vessel for combining the premix composition and water; or
(v) a single or multi-use vessel or apparatus suitable for sinus irrigation.
